(19) United States
(12) Patent Application Publication
(10) Pub. No.: US 2010/0023018 A1
(43) Pub. Date: Jan. 28, 2010

(54) Spinous process fixated bilateral drilling guide

(76) Inventor: Charles S. Theofilos, Palm Beach Gardens, FL (US)

Correspondence Address: MCHALE & SLAVIN, P.A., 2855 PGA BLVD, PALM BEACH GARDENS, FL, 33410, US

(21) Appl. No.: 12/507,566

(22) Filed: Jul. 22, 2009

Related U.S. Application Data
(60) Provisional application No. 61/083,014, filed on Jul. 23, 2008.

(51) Int. Cl. A61B 17/58

(52) U.S. Cl. 606/96

(57) ABSTRACT

The application describes a bilateral drilling and screw placement guide adapted for fixation to the spinous process of a vertebral body. The positioning and surgical guiding instrument is adapted for use during a spinal surgical procedure in conjunction with a drilling tool, fastening device, e.g. a pedicle screw, K-wire or the like. The guide includes an engagement device for attachment to a region of the spinous process, to which an adjustably attached support member is affixed. This allows for immobilizing the guide upon the spinous process anatomical landmark, in a specific orientation with respect thereto. The guide includes a bilaterally adjustable drill guide assembly for precise anatomical positioning of the drill and screw placement guides bilaterally about the sagittal, axial and coronal planes so as to enable the defining of a plurality of drilling axes extending toward the vertebral body. The device further provides for adjustment to account for anatomical variations in width along the axial plane, and includes pointing and angular positioning functionality to insure repeatable and reliable pilot hole and screw placement along a plurality of angles.

### REFERENCE TO RELATED APPLICATIONS

The present application claims the benefit of priority of U.S. provisional application Ser. No. 61/083,014, filed Jul. 23, 2008, the contents of which are incorporated herein by reference.

### FIELD OF THE INVENTION

This invention relates to drilling guides for safely and accurately establishing drilling and screw placement positions within the spinal anatomy; particularly to a guide which utilizes the spinous process as an anatomical landmark; and most particularly to a bilateral drilling guide which is adapted for fixation to the spinous process, and is constructed to provide repeatable and reliable guidance for placement of drills or screws along plural spinal trajectories.

### BACKGROUND OF THE INVENTION

The drilling of holes in bones in order to accommodate fastening devices used to anchor implants within a patient's body has become fairly commonplace. In the field of spinal surgery, for example, the use of pedicle screws to anchor internal instrumentation systems to the spinal column of patients for correcting a variety of spinal disorders provides many advantages over other anchoring devices, such as hooks, but the risks associated with their implantation into the vertebral column of the patients have limited their universal acceptance, and remain a cause of concern for both the patient and physician.

The generally accepted technique for inserting a pedicle screw requires the physician to prepare a pilot hole through the pedicle before inserting the screw therein. Typically, the selection of the insertion point can be made from anatomical techniques or a preoperative CT scan. The preoperative CT scan is useful for determining the angle of insertion, as well as for screw length and diameter. Additionally, it is also known to use intra-operative X-rays or fluoroscopic guidance before pedicle hole preparation to determine the appropriate size and placement of the pedicle screws. After an appropriate pedicle has been selected, an initial small puncture through the posterior cortex can be made to mark the insertion point and to facilitate the drilling procedure. Then, the pilot hole is usually made with a drill bit or a pedicle probe.

Prior to insertion of the screw in the pilot hole, the latter may be manually probed in order to check for violation of the pedicle walls. The screw length can be determined by the use of a calibrated probe inserted into the pedicle pilot hole. Intra-operative X-rays or fluoroscopy are also generally useful to verify the accuracy of the pilot hole or the screw.

In an effort to minimize malpositioning of the pilot hole or ultimate screw placement, various methods have been developed. For example, electrical methods and saline injection technique have been developed to confirm the correct path of the pilot hole prior to pedicle screw insertion. Computer-assisted guidance systems have also been developed in order to visually track the intra-operative position of the vertebra during pedicle screw insertion.

While prior cadaveric research had shown that the lateral mass and facets are landmarks to determine the initial starting point for lateral mass screws and further indicated that the optimum screw trajectory was 30 degrees lateral and 15 degrees cephalad, the research conducted by Chin et al, (J Spinal Disord Tech, February 2006; 19(1):18-21) concluded that what was missing was an intraoperative landmark to guide the trajectory for drilling according to these angles. Chin et al hypothesized that spinous processes can be used to guide the trajectory for lateral mass screw placement, and conducted research measuring the lateral and cephalocaudad angles for each lateral mass from C3 to C6 while using the spinous processes of the adjacent three caudad vertebrae at each level to guide the starting trajectories. As a result of this work, they concluded that the spinous processes can be an accurate local anatomic guide for lateral mass screw trajectory and will allow greater safety while drilling before performing laminectomies.

Therefore, if a drilling and screw placement guide could be provided that fixated upon the spinous process, allowed for adjustment so as to have bilateral utility about the sagittal and coronal planes, and be infinitely adjustable in all angles, e.g. the cephalocaudad angle, lateral angle, medial angle, etc., a long-felt need in the art would be satisfied.

### Prior Art

U.S. Pat. No. 4,907,577, issued to Wu discloses a spinal transpedicle drill guide having an I-shaped body, a guiding base and a positioning base which are slidable in a transversal direction on the I-shaped body to prevent deviation of the drilling direction in the transversal plane of the vertebra of the pedicle. However, this guide does not allow for adjustment of the drilling path in the lateral plane of the vertebra.

U.S. Pat. No. 5,163,940 to Bourque is directed toward a surgical drill guide having an arcuate support beam and a locator probe, and is adapted to hold a cannulated drill sleeve. This device is not useful for fixation to a spinous process.

Tromanhauser et al (U.S. Pat. No. 7,060,068) and Schneiderman (U.S. Pat. No. 6,547,795) each teach screw placement guides for a vertebral member.

These references fail to teach or suggest attachment to the spinous process, nor are they adapted for repeatable and reliable alignment to a plurality of angles to enable accurate preparation and placement of fasteners along various spinal aspects.

### SUMMARY OF THE INVENTION

In accordance with the present invention there is provided a bilateral drilling and screw placement guide adapted for fixation to the spinous process of a vertebral body. The positioning and surgical guiding instrument is adapted for use in conjunction with a drilling tool. The guide includes an engagement device for attachment to a region of the spinous process, to which an adjustably attached support member is affixed. This allows for immobilizing the guide upon the spinous process anatomical landmark, in a specific orientation with respect thereto. The guide includes a bilaterally adjustable drill guide assembly for precise anatomical positioning of the drill and screw placement guides bilaterally about the sagittal and coronal planes so as to enable the defining of a plurality of drilling axes extending toward the vertebral body. The device further provides for adjustment to account for anatomical variations in width along the axial plane, and includes pointing and angular positioning functionality which provides for repeatable and reliable pilot hole and screw placement along a plurality of angles about the sagittal and coronal planes.

In an illustrative but non-limiting embodiment the bilaterally positionable drilling and screw placement guide includes a clamp and support assembly adapted for removable engagement with a spinous process of a vertebral body; wherein the support assembly has a distal end incorporating a clamp assembly and a proximal end having a baseplate in rotatable engagement with a bilaterally positionable extension which enables the bilaterally positionable extension to be rotated 180° and thereby be positionable perpendicular to the clamp and support assembly on either side of the body, and further includes a guide and support member for alignment of at least one drill bushing, and at least one bushing fixation support for fixedly engaging at least one drill bushing thereby providing an instrument which enables accurate and repeatable drilling and screw placement within a vertebral body within the axial, coronal and sagittal planes. Optionally, the device may include a pointing device in parallel alignment with the clamp and support assembly for assisting in positioning of the drilling and screw placement guide relative to the vertebral body, and at least one protractor device adapted for rotatable and removable engagement upon the guide and support assembly, and via its rotatable mounting, is adapted for measurement of angles residing within the axial, coronal or sagittal planes.

In an alternative embodiment the support assembly distal end is constructed and arranged for rotatable engagement with the clamp assembly proximal end which permits adjustments to be made for anatomic anomalies in the spinous process.

Accordingly, it is a primary objective of the instant invention to provide a surgical guiding instrument which can be used to properly orient a drilling axis and enable screw placement subsequent to fixation upon the spinous process.

It is a further objective of the instant invention to provide a surgical guiding instrument which is adapted to allow for safe insertion of a screw into a bone.

It is yet another objective of the instant invention to provide a surgical guiding instrument which is relatively simple and economical to manufacture.

It is a still further objective of the instant invention to provide a device and method for safely preparing a pilot hole in a pedicle of a selected vertebra of a patient's spinal column.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

Having thus generally described the nature of the invention, reference will now be made to the accompanying drawings, showing by way of illustration a preferred embodiment thereof, and in which:

FIG. 1 is a perspective view of one embodiment of the invention mounted upon a spinous process;

FIG. 2 is an orthogonal frontal view of the embodiment of FIG. 1;

FIG. 3A is a partial exploded view of the clamping mechanism and support assembly of FIG. 1;

FIG. 3B is a side exploded view of the clamping mechanism and support assembly of FIG. 1;

FIG. 4A is a top view of one embodiment of a bilaterally positionable extension;

FIG. 4B is a side view of one embodiment of a bilaterally positionable extension;

FIG. 4C is a left end view of one embodiment of a bilaterally positionable extension;

FIG. 4D is a right end view of one embodiment of a bilaterally positionable extension;

FIG. 5A is a top view of the telescoping guide and support member;

FIG. 5B is a right end view of the telescoping guide and support member showing the pointer device extending upwardly;

FIG. 6A is a front view of the guide fixation assembly;

FIG. 6B is a right end view of the guide fixation assembly;

FIG. 7A is a top view of one embodiment of a drill bushing;

FIG. 7B is a side view of the drill bushing of FIG. 7A;

FIG. 8 is a perspective view of an alternative embodiment of the invention mounted upon a spinous process;

FIG. 9 is an orthogonal frontal view of the embodiment of FIG. 8;

FIG. 10A is a frontal view of an alternative embodiment of the bilaterally positionable extension;

FIG. 10B is a left end view of an alternative embodiment of the bilaterally positionable extension;

FIG. 10C is a right end view of an alternative embodiment of the bilaterally positionable extension;

FIG. 11A is a front view of a polyaxial bushing support;

FIG. 11B is a side view of the polyaxial bushing support of FIG. 11A;

FIG. 12A is front view of an embodiment of an adjustable drill bushing assembly;

FIG. 12B is right side view of an embodiment of an adjustable drill bushing assembly of FIG. 12A;

FIG. 12C is a section view taken along line 12C-12C of FIG. 12A;

FIG. 13A is a front view of a rotatable extension/bushing support;

FIG. 13B is a right side view of the rotatable extension/bushing support of FIG. 12A;

FIG. 14A is a front view of an anatomical landmark pointing device;

FIG. 14B is a side view of an anatomical landmark pointing device;

FIG. 15 is a perspective view of an embodiment of the invention for mounting upon a spinous process using a minimally invasive surgical technique;

FIG. 16 is an orthogonal frontal view of the embodiment of FIG. 15.

### DETAILED DESCRIPTION OF THE INVENTION

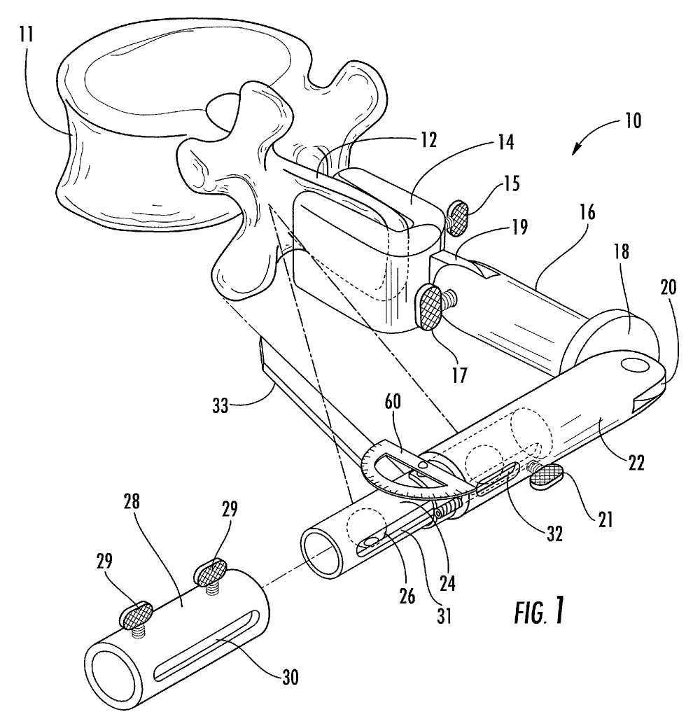

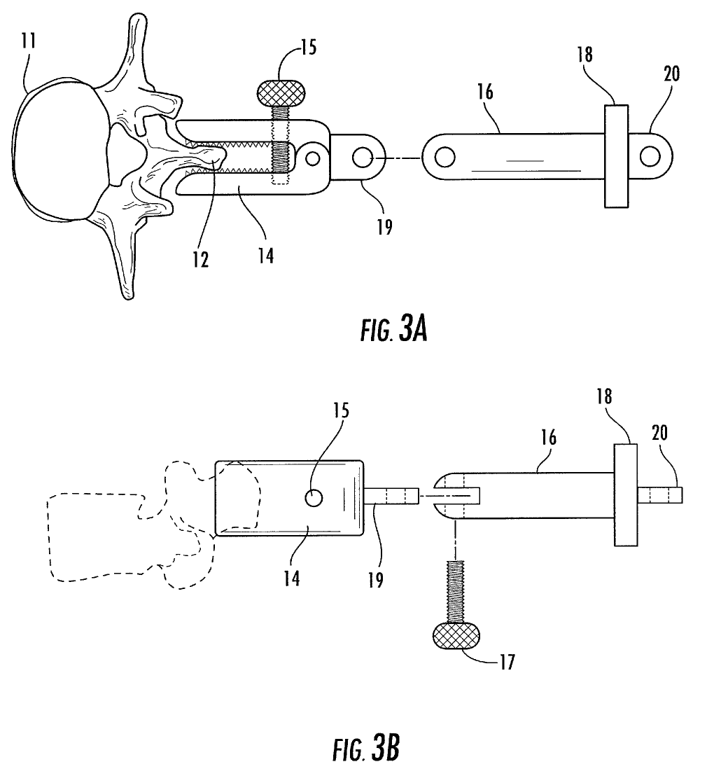

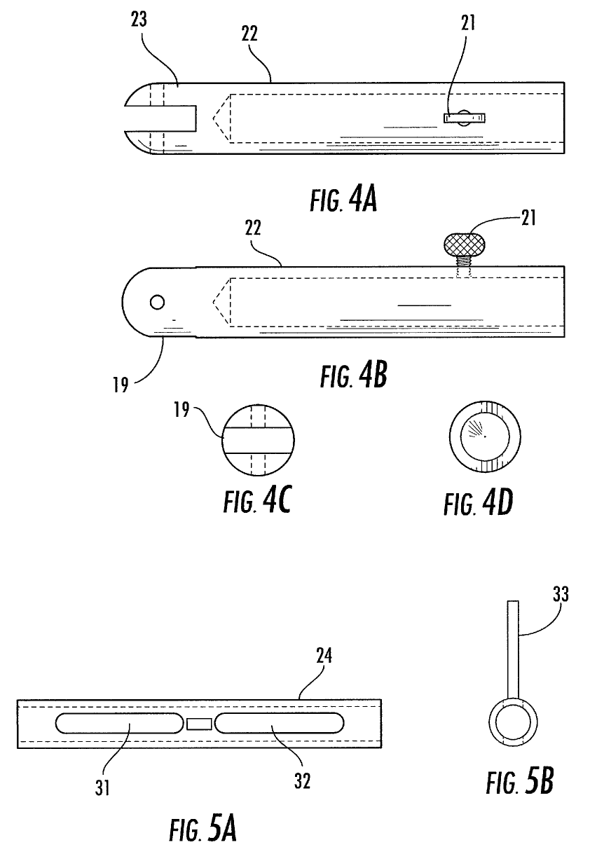

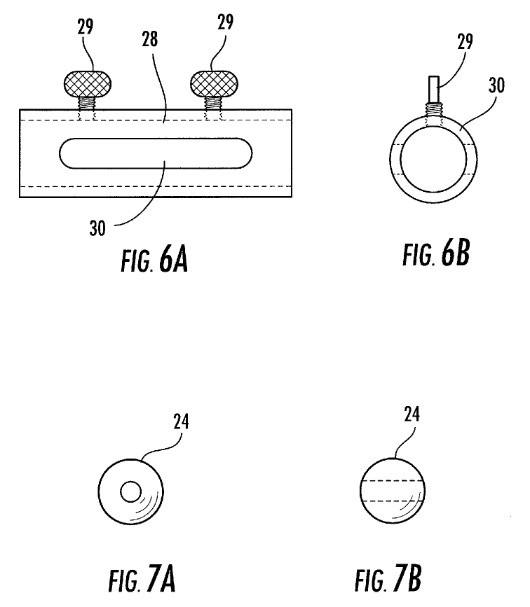

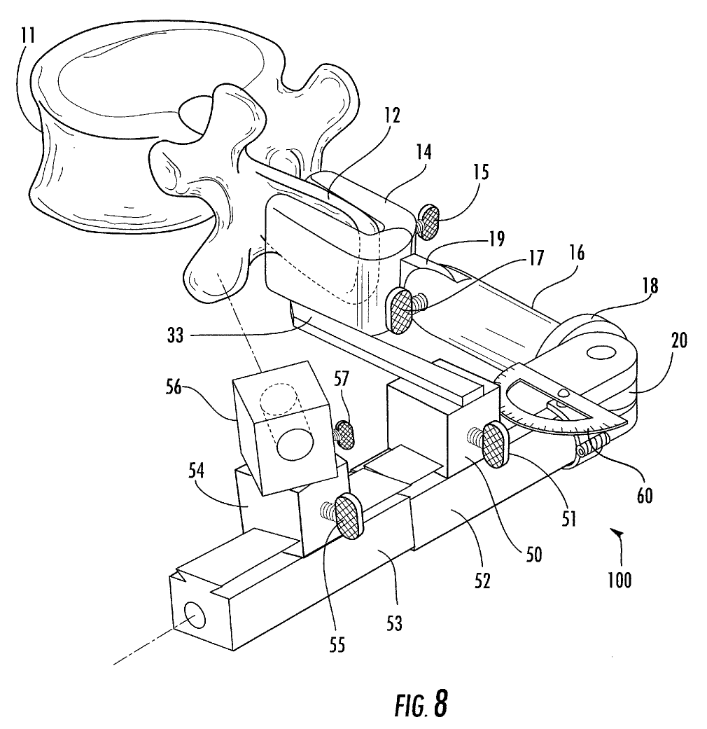

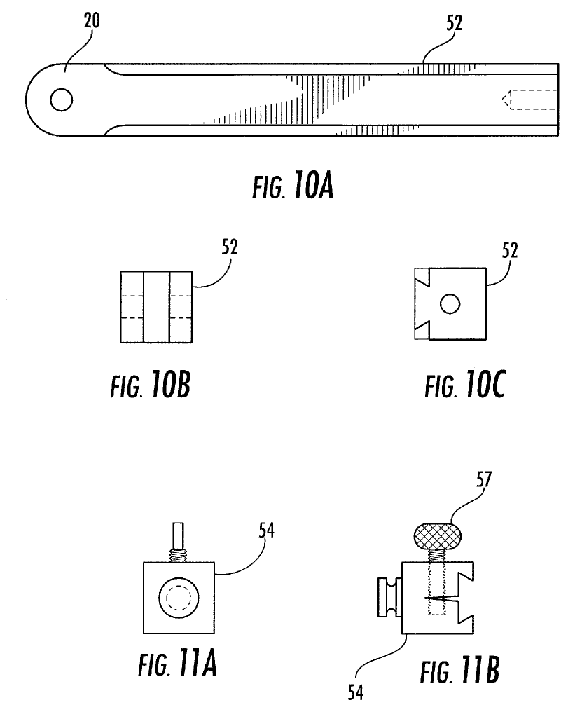

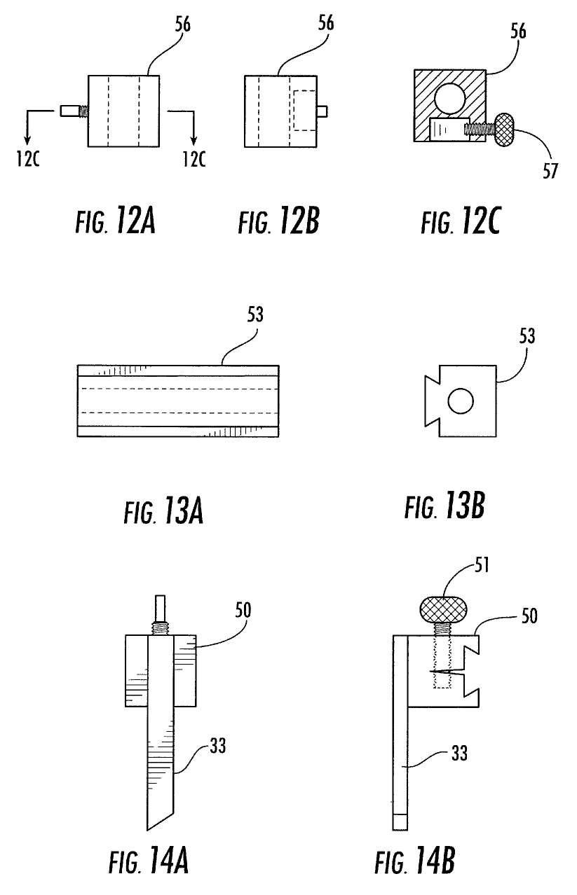

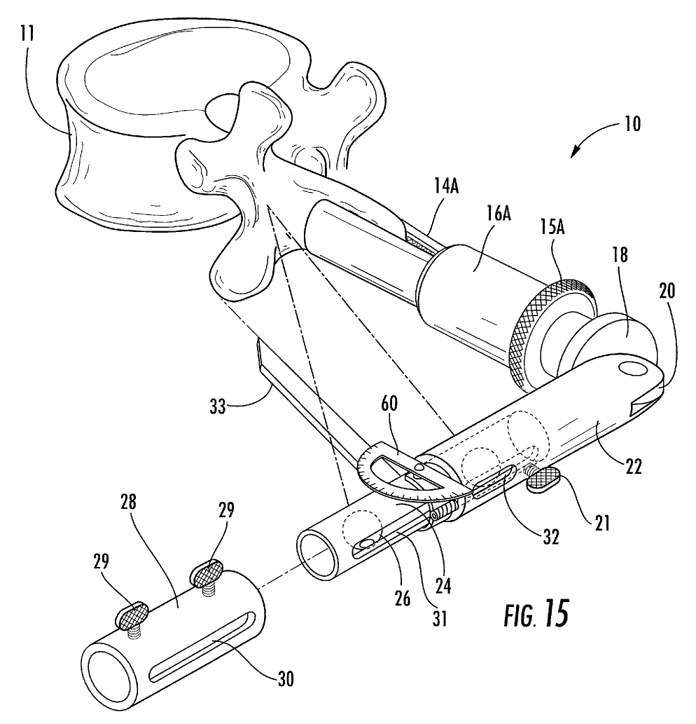

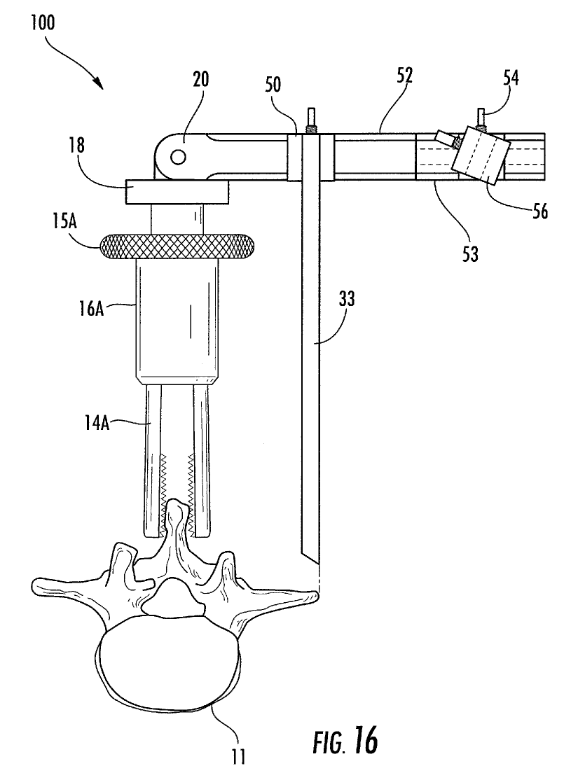

The invention will now be explained in detail, by reference to FIGS. 1-14, wherein like numbers refer to like components throughout.

Now referring to FIG. 1, a first embodiment of the bilaterally positionable drilling and screw placement guide, generally referred to as 10, is illustrated fixedly engaged with spinous process 12 which extends from vertebral body 11. Clamp assembly 14 is adapted to fixedly engage the spinous process 12 and be tightened via thumbscrew 15. Tab 19 extends from clamp 14 and couples with support assembly 16. This allows for the surgeon to make minor adjustments to the support mechanism to allow for anatomic anomalies in the spinous process 12. At the uppermost portion of support assembly 16 a base plate 18 is situated, and protruding therefrom is another tab 20, which is inserted into and in rotatable engagement with bilaterally positionable extension 22. This assemblage permits the bilaterally positionable extension 22 to be rotated 180°, thereby permitting the device to be used on either side of the body, while having the extension 22 resting upon base plate 18 causes the bilaterally positionable extension 22 to remain in perpendicular orientation to the support assembly 16, thereby insuring that the assembly 16 and pointing device 33 remain parallel at all times. Telescoping guide and support member 24 may contain one or more drill bushings 26 which are accessible and positionable through slot(s) 31. If steeper angulation is required, slot 32 provides access to an alternative drill bushing 26, which may be fixedly engaged by pressure from thumbscrew 21. In order to accurately determine the angulation in the pertinent planes, the physician may attach protractor device 60 which is illustrated as rotatably attached to a spring-clip or the like for removable engagement upon any face of elements 24, 28 or 22, and via its rotatable mounting, is adapted for measurement of angles residing within the axial, coronal or sagittal planes. One exemplary, albeit non-limiting means for locking in the angulation of the drill bushings 26 is by provision of a guide fixation assembly 28 which is adapted for slidable and overlying engagement with member 24 such that tightening of set screws 29 results in sufficient compression of member 24 to immobilize bushings 26. Access to bushings 26 is obtained through slot 30.

With reference to FIG. 2, a frontal view of device 10 is illustrated when attached to a spinous process 12, illustrating how a desired angulation and positioning can be accomplished.

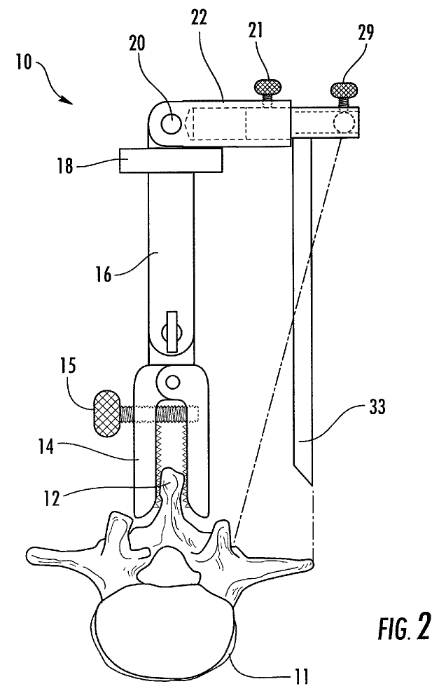

Referring now to FIGS. 3A, 3B, 4A, 4B, 4C, 4D, 5A, 5B, 6A, 6B, 7A and 7B, the individual components of the device of FIG. 1 are further illustrated in varying views so as to fully disclose the concepts of the invention. One of ordinary skill will readily understand that these are exemplary and non-limiting embodiments and the invention contemplates alterations in shapes, structures, guides and fastening members which do not depart from the spirit and scope of the invention.

Referring to FIG. 8, an alternative embodiment 100 is illustrated. In this embodiment the forward edge of bilaterally positionable extension element 52 is dove-tailed, as is that of rotatable extension/bushing support 53. This allows for slidable engagement of polyaxial bushing support 54 and adjustable drill bushing assembly 56 which, by virtue of their attachment to rotatable extension/bushing support 53, permit reliable and repeatable rotation and traversal within the sagittal, coronal and axial planes. By first aligning pointing device 33, which is affixed to slidable element 50, the axial spacing can be delineated. Following this, one degree of adjustment may be affected by virtue of the rotation of the rotatable extension/bushing support 53, while a second degree of adjustment is affected by virtue of rotation and/or traversal of the adjustable drill bushing assembly 56. It is contemplated that plural polyaxial bushing supports 54 and adjustable drill bushing assemblies 56 may be utilized, and that these components may alternatively be positioned upon bilaterally positionable extension element 52, adjacent to, or instead of slidable element 50, in order to enable steeper angulations, as may be required.

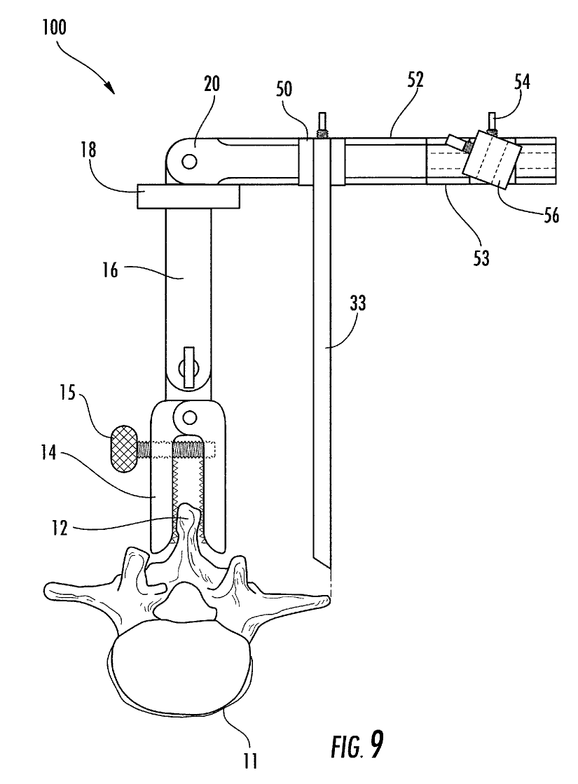

Referring to FIGS. 9, 10A-10C, 11A, 11B, 12A-12C, 13A, 13B 14A and 14B the individual components of the device of FIG. 8 are further illustrated in varying views so as to fully disclose the concepts of the invention. One of ordinary skill will readily understand that these are exemplary and non-limiting embodiments and the invention contemplates alterations in shapes, structures, guides and fastening members which do not depart from the spirit and scope of the invention.

With reference to FIG. 15, an alternative embodiment which parallels that of FIG. 1 is illustrated. The first embodiment as illustrated in FIG. 1 is designed for invasive surgical procedures where the spinous process is exposed. When a minimally invasive surgical technique is desirable, the bilaterally positionable drilling and screw placement guide, generally referred to as 10, is again illustrated as fixedly engaged with spinous process 12 which extends from vertebral body 11. In this instance the clamping and supporting assembly is in the form of a collet clamp 14A and cap 16A. The collet clamp 14A is formed of a material, e.g. a spring steel or the like polymeric material having spring-like properties, which is normally biased outward from a central longitudinal axis, and has an external taper. Cap 16A is slidably engaged about the collet clamp 14A and is designed to be drawn upwardly or downwardly about the collet clamp so as to apply or release inwardly directed pressure upon the jaws of the collet clamp 14A. As illustrated, this may be accomplished by providing external clamping adjustment, for example in the form of rotation of knurled wheel 15A, which is coupled to a threaded rod (not shown) which enables longitudinal reciprocal movement of the cap 16A along the collet chuck 14A. In an alternative embodiment, it is contemplated that the function of knurled wheel assembly 15A could be accomplished by replacement with a pivoting drawbar assembly (not shown) for providing a lever action type of locking mechanism. This allows for the surgeon to make minor adjustments to the support mechanism to allow for anatomic anomalies in the spinous process 12. At the uppermost portion of cap 16A a base plate 18 is situated, and protruding therefrom is another tab 20, which is inserted into and in rotatable engagement with bilaterally positionable extension 22. This assemblage permits the bilaterally positionable extension 22 to be rotated 180°, thereby permitting the device to be used on either side of the body, while having the extension 22 resting upon base plate 18 causes the bilaterally positionable extension 22 to remain in perpendicular orientation to the support assembly 16, thereby insuring that the assembly 16 and pointing device 33 remain parallel at all times. Telescoping guide and support member 24 may contain one or more drill bushings 26 which are accessible and positionable through slot(s) 31. If steeper angulation is required, slot 32 provides access to an alternative drill bushing 26, which may be fixedly engaged by pressure from thumbscrew 21. In order to accurately determine the angulation in the pertinent planes, the physician may attach protractor device 60 which is illustrated as rotatably attached to a spring-clip or the like for removable engagement upon any face of elements 24, 28 or 22, and via its rotatable mounting, is adapted for measurement of angles residing within the axial, coronal or sagittal planes. One exemplary, albeit non-limiting means for locking in the angulation of the drill bushings 26 is by provision of a guide fixation assembly 28 which is adapted for slidable and overlying engagement with member 24 such that tightening of set screws 29 results in sufficient compression of member 24 to immobilize bushings 26. Access to bushings 26 is obtained through slot 30.

With reference to FIG. 16, a frontal view of device 100 (see FIGS. 8 and 9) is illustrated when attached to a spinous process 12, via a minimally invasive clamping system as illustrated by elements 14A, 15A and 16A, illustrating how a desired angulation and positioning can be accomplished.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bilaterally positionable drilling and screw placement guide adapted for removable engagement with a spinous process of a vertebral body comprising:
a clamp assembly and support assembly adapted for removable engagement with a spinous process of a vertebral body;
said support assembly having a distal end incorporating said clamp assembly and a proximal end having a baseplate in rotatable engagement with a bilaterally positionable extension which enables said bilaterally positionable extension to be rotated so as to be positionable substantially perpendicular to said clamp and support assembly on either side of the vertebral body;
a guide and support member for alignment of at least one drill bushing; and
at least one bushing fixation support for fixedly engaging said at least one drill bushing;
whereby accurate and repeatable drilling and screw placement within said vertebral body is enabled within the axial, coronal and sagittal planes.

2. The bilaterally positionable drilling and screw placement guide in accordance with claim 1 wherein:
said support assembly distal end is constructed and arranged for rotatable engagement with said clamp assembly proximal end;
said rotatable engagement adjustable for anatomic anomalies in the spinous process.

3. The bilaterally positionable drilling and screw placement guide in accordance with claim 1 wherein:
said guide and support member are constructed and arranged for slidable adjustment for positioning said at least one drill bushing fixation support with respect to said vertebral body.

4. The bilaterally positionable drilling and screw placement guide in accordance with claim 1 wherein:
said clamp and support assembly includes clamping adjustment external of the body for securing said bilaterally positionable drilling and screw placement guide to the spinous process of a vertebral body.

5. The bilaterally positionable drilling and screw placement guide in accordance with claim 1 wherein:
said bushing fixation support for fixedly engaging said at least one drill bushing includes a guide fixation assembly adapted for slidable and overlying engagement with said guide and support member which is constructed and arranged to provide sufficient compression of said clamp and support assembly to immobilize said at least one drill bushing.

6. The bilaterally positionable drilling and screw placement guide in accordance with claim 1 further including:
a pointing device in parallel alignment with said clamp and support assembly for assisting in positioning of the drilling and screw placement guide relative to the vertebral body.

7. The bilaterally positionable drilling and screw placement guide in accordance with claim 1 further including:
at least one protractor device adapted for rotatable and removable engagement upon said guide and support assembly, and via its rotatable mounting, is adapted for measurement of angles residing within the axial, coronal or sagittal planes.

8. The bilaterally positionable drilling and screw placement guide in accordance with claim 1 wherein:
said bushing fixation support is polyaxial.
